(19) United States
(12) Patent Application Publication
(10) Pub. No.: US 2005/0124959 A1
(43) Pub. Date: Jun. 9, 2005

(54) Absorbent articles with asymmetric protective wing portions

(75) Inventors: Glory F. Alcantara, Neenah, WI (US); MeeWha Lee, Appleton, WI (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 10/731,735

(22) Filed: Dec. 9, 2003

(51) Int. Cl. A61F 13/15; A61F 13/20

(52) U.S. Cl. 604/385.04; 604/358

(57) ABSTRACT

An absorbent article, such as a feminine care product, includes a pad component having a liquid permeable top cover, a generally liquid impermeable baffle, and an absorbent structure disposed between the top cover and outer cover. Protective wing portions extend laterally outward from lateral sides of the pad component and have dimensions so as to wrap at least partially around the crotch portion of a wearer's undergarment. The wing portions extend longitudinally along both sides of a transverse centerline of the pad and have an asymmetric shape with an overlap region disposed forward of the transverse centerline.

### FIELD OF THE INVENTION

The present invention relates generally to the field of disposable absorbent articles, and more particularly to a feminine care article having laterally extending protective wings.

### BACKGROUND

Disposable absorbent articles intended to be worn in the crotch portion of an undergarment are well known. The basic form of such absorbent articles typically includes a body-side liquid-permeable top cover, a garment-side liquid-impermeable baffle, and an absorbent core positioned between the top cover and the baffle. Such absorbent articles are now in wide use as sanitary napkins, panty shields, panty liners, and adult incontinence pads. While this invention is directed to all such products, for purposes of simplification, the invention will be described with reference to a sanitary napkin.

In an effort to provide enhanced protection, it is known to provide conventional disposable absorbent articles with laterally extending flaps or wing portions intended to be folded around the edges of the wearer's undergarment. The wings offer some functional improvements and advantages over articles without such devices. First, the wings protect the edges of the wearer's undergarment from being soiled by bodily fluids. Second, the wings help to stabilize the sanitary napkin from shifting out of place, especially when the panels are affixed to the underside of the garment. Typically, each wing is either integral with the cover and/or the baffle or can be fashioned from separate pieces of material and permanently adhered or otherwise attached to the article so as to extend laterally from the sides of the article.

Conventional wing designs are generally symmetric and define a laterally outboard region that extends across the crotch region of the wearer's undergarment and attaches to the undergarment or to the opposite wing. Because of the their symmetric design and placement relative to the longitudinal dimension of the article, the attaching regions extend laterally along a transverse centerline of the absorbent article and, thus, attach around the center portion of the undergarment's crotch region. However, this attachment location may be problematic for certain users.

For example, the middle or inner crotch region of the undergarment to which the conventional wings are attached, is continually distorted, twisted and stretched due to the dynamics of the wearer. As a result, the wing attachment mechanism is subjected to substantial stresses and frequently detaches in use causing the wings and article to shift.

Also, the wings may be problematic in their placement and retention in the middle crotch area of the undergarment, particularly when the undergarment remains on the wearer and is simply lowered by the wearer prior to placement of the article. Usually, when the wearer is ready to use the absorbent article, a release liner is peeled from an adhesive on the baffle side of the article and the article is then pressed into the crotch region of the undergarment. The wings are then folded around the crotch region and secured on the underside of the undergarment, either affixed to the garment-facing side of the undergarment or to each other. This process requires the wearer to reach the lowermost portion of the undergarment and manipulate the wings in an awkward location and generally without being able to see the wings. Once the adhesive has been exposed and the article placed into the crotch region of the undergarment, it is generally not practical to remove and reposition the article.

It is also the case that the centrally disposed asymmetric wings are not optimally positioned for all styles of undergarments. For example, the crotch region of many undergarment styles taper significantly from the waist regions with the narrowest section of the crotch region to which the wings are attached actually being narrower than the pad component of the article. Attachment of the wings in this location serves little purpose.

The present invention provides an absorbent article with a unique wing configuration that addresses the drawbacks of conventional designs described above.

### SUMMARY

Objects and advantages of the invention will be set forth in part in the following description, or may be obvious from the description, or may be learned through practice of the invention.

Although the present invention has particular usefulness in the field of feminine care articles such as panty shields, liners and sanitary napkins, it should be appreciated that any manner of personal care absorbent article may benefit from the invention, including incontinence articles, and the like. All such uses are within the scope and spirit of the invention. For ease of description only, the working environment of the invention is assumed to be feminine care sanitary napkins.

In a particular embodiment of the invention, an absorbent article is provided for placement in the crotch region of an undergarment. The article includes a pad component having conventional features such as a generally liquid permeable top cover, a generally liquid impermeable outer cover or baffle, and an absorbent structure disposed between the top cover and baffle. The invention is not limited to any particular pad shape or configuration. Protective wing portions extend laterally outward from lateral sides of the pad component, the wing portions having dimensions so as to wrap at least partially around the crotch region of the wearer's undergarment. The wing portions have laterally outboard regions that overlap when the wings are folded around the crotch region of the wearer's undergarment. The wings may be portions of individual flap or panel structures that are adhered or otherwise attached to the baffle side of the pad. In an alternate embodiment, the wing portions may be components of a unitary flap or panel component that is attached to the baffle. In another embodiment, the wing portions may be defined as lateral extensions of the baffle material.

The wing portions extend longitudinally along both sides of a transverse centerline of the pad and have an asymmetric shape such that the overlap regions are disposed forward of the transverse centerline of the pad. For example, the wing portions may extend along about two-thirds of the lateral sides of the pad, and may extend equally on each side of the transverse centerline with the overlap region disposed entirely at the forward part of the pad (forward of the transverse centerline). With this configuration, the article may be placed in the desired middle or lowermost part of the undergarment's crotch region with the wing portions being secured closer to the front waist region of the undergarment. The wing portions may be thought of as forwardly biased on the article. This configuration offers substantial benefits. For example, the wings are attached to the undergarment or to each other at a wider part of the crotch region, thus providing a more secure attachment. Also, the location of the wings provides a visual indication to the wearer of the correct orientation of the article for use. The asymmetric shape also allows for variable fastening or fold angles of the wing portions with respect to the lateral sides of the article while retaining the overlap regions at the forward part of the article. For example, the fold lines may be angled away from the lateral sides of the pad from a forward most point of the fold lines to a rearward most point. In alternate embodiments, the fold lines may be angled towards the lateral sides of the pad from a forward most point of the fold lines to a rearward most point. This flexibility permits the user to easily customize the article to various styles and cuts of undergarments. In a particular embodiment, at a maximum angled position of the fold lines, the overlap regions are maintained at least partially overlapping and remain entirely on the forward side of the transverse centerline of the pad.

In one particular embodiment, the asymmetric wing portions have an outboard edge that tapers generally from the wing's respective overlap region towards the lateral side of the pad. For example, the overlap region may be disposed at a forward most position on the wing portion and be defined by a forward most edge that extends generally perpendicular to the lateral side of the pad such that the wing portion assumes an overall right triangle-type configuration. In an alternate embodiment, the forward most edge may extend at an angle from the lateral side of the pad towards the forward longitudinal end of the pad.

Aspects of the invention will described below in greater detail by reference to particular embodiments, examples of which are illustrated in the figures.

### BRIEF DESCRIPTION OF THE FIGURES

FIG. 1 is a perspective and partial cut-away view of an absorbent article according to the invention.

FIG. 2 is a perspective view of the absorbent according to FIG. 1 placed in an undergarment.

FIGS. 3A through 3D are garment facing planar views of the absorbent article of FIG. 1 as it would be attached at varying angular orientations on the crotch region of an undergarment.

FIG. 4 is a garment facing planar view of an absorbent article illustrating alternate wing portion configurations in phantom lines.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Referring to the Figures, in which like numerals indicate like parts throughout the several views, embodiments of a disposable absorbent article 10 are depicted. For purposes of illustration only, the disposable absorbent article 10 is exemplified as a sanitary napkin. Typically, a sanitary napkin is worn by a female to absorb body fluids, such as menses, blood, urine and other body excrements discharged during a menstrual period. The term “disposable”, as used herein, means that the absorbent article is discarded after a single use and is not intended to be laundered for subsequent reuse.

One skilled in the art will readily understand the adaptability of the invention to other personal care and health care articles, such as, for example, panty liners, adult incontinence garments and the like that use a securement panel to position the article relative to a wearer's undergarment. It should thus be appreciated that the invention is in no way limited to sanitary napkins in particular, or to feminine care articles in general.

Referring to the figures in general, the absorbent article 10 according to the invention includes a primary pad component 18. It should be appreciated that the shape of the pad component 18 depicted in the figures is for illustration purposes only, and that any shape or style of pad component 18 is within the scope and spirit of the invention. In use, the pad 18 is longitudinally oriented along a longitudinal centerline 21, and has a transverse centerline 20 defined about midway between opposite longitudinal ends 24, 26. The pad 18 has a generally liquid permeable top cover 28, a generally liquid impermeable outer cover or “baffle” 30, and an absorbent structure 32 disposed between the top cover 28 and baffle 30. The top cover 28 and baffle 30 are sealed together at their peripheral edges utilizing known techniques, such as, for example, gluing, crimping, hot-sealing or the like, the sealed edges defining an overall sealed peripheral edge 33. The pad 18 may take on various shapes, but will generally have opposite lateral sides 22 and longitudinal ends 24 and 26, with the end 24 being the forward or front end of the pad 18. In the illustrated embodiments, the pad 18 has a well-known elongated race-track shape with generally parallel sides 22. In an alternate embodiment, the sides 22 may define a generally hourglass shape. Various other geometries of absorbent articles, including feminine care articles, are well known to those skilled in the art, and all such embodiments are within the scope and spirit of the invention.

The absorbent article 10, particularly the pad 18, is desirably provided with sufficient capacity to absorb and retain the intended amount and type of bodily exudate(s). The absorbent capacity is provided by the fluid retentive absorbent structure 32. The absorbent structure 32 can be any structure or combination of components which are generally compressible, conformable, non-irritating to a wearer's skin, and capable of absorbing and retaining liquids and certain body wastes. For example, the structure 32 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In a particular embodiment, the absorbent web material is a matrix of cellulosic fluff, and may also include superabsorbent hydrogel-forming particles. The cellulosic fluff may comprise a blend of wood pulp fluff. One preferred type of fluff is identified with the trade designation NB 416, available from Weyerhaeuser Corp., and is a bleached, highly absorbent wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art.

The absorbent structure 32 can contain superabsorbent materials which are effective in retaining body fluids. As a general rule, the superabsorbent material is present in the absorbent web in an amount of from about 0 to about 90 weight percent based on total weight of the web. Superabsorbents have the ability to absorb a large amount of fluid in relation to their own weight. Typical superabsorbents used in absorbent articles, such as sanitary napkins, can absorb anywhere from 5 to 60 times their weight in body fluids. Superabsorbent materials are well known in the art and can be selected from natural, synthetic, and modified natural polymers and materials.

The absorbent web material may also be a coform material. The term “coform material” generally refers to composite materials comprising a mixture or stabilized matrix of thermoplastic fibers and a second non-thermoplastic material. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes.

The absorbent web material utilized in the absorbent structure 32 is also selected so that the individual absorbent structure possesses a particular individual total absorbency depending on the intended article of use. For example, for infant care products, the total absorbency can be within the range of about 200-900 grams of 0.9 wt % saline, and can typically be about 500 g of saline. For adult care products, the total absorbency can be within the range of about 400-2000 grams of saline, and can typically be about 1300 g of saline. For feminine care products, the total absorbency can be within the range of about 7-50 grams of menstrual fluid, and can typically be within the range of about 30-40 g of menstrual fluid.

The absorbent structure 32 may be a multi-component and may include, for example, an intake layer or transfer delay layer in combination with the underlying absorbent web. Such configurations are well known to those skilled in the art.

The fluid permeable top cover 28 has an outwardly facing surface that may contact the body of the wearer and receive bodily exudate(s). The top cover 28 desirably is made of a material which is flexible and non-irritating to the wearer. As used herein, the term “flexible” is intended to refer to materials which are compliant and readily conform to the bodily surface(s) with which such materials are in contact, or materials which respond by easily deforming in the presence of external forces.

The top cover 28 is provided for comfort and conformability and functions to direct bodily exudate(s) away from the body, through the top cover 28 and toward the absorbent structure 32. The top cover 28 should retain little or no liquid in its structure so that the cover provides a relatively comfortable and non-irritating surface next to the tissues within the vestibule of a female wearer. The top cover 28 can be constructed of any woven or nonwoven material which is easily penetrated by bodily fluids which contact the surface of the cover. Examples of suitable cover materials include rayon, bonded carded webs of polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated film webs and net material can also be used. The cover may be apertured to increase its fluid intake capacity. A specific example of a suitable cover material is a bonded carded web made of polypropylene and polyethylene such as that used as cover stock for KOTEX® pantiliners and obtainable from Sandler Corporation, Germany. Other examples of suitable materials are composite materials of polymer and nonwoven fabric materials. The composite materials are typically in the form of integral sheets generally formed by the extrusion of a polymer onto a web of spunbonded material. The fluid permeable cover 28 can also contain a plurality of apertures formed therein which are intended to increase the rate at which bodily fluid(s) can penetrate through the cover and into the absorbent structure 32.

The top cover 28 may also be embossed with any desired embossing pattern to define embossed channels. Embossing techniques are well known to those skilled in the art. An embossing pattern not only creates an aesthetically pleasing surface, the channels facilitate intake of menses fluid. Menses will tend to flow along the densified edges of the channels rather than pool on contact points of the top cover 28.

The top cover 28 can be maintained in secured relation with the absorbent structure 32 by bonding all or a portion of the adjacent surfaces to one another. A variety of bonding methods known to one of skill in the art can be utilized to achieve any such secured relationship. Examples of such methods include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The baffle 30 may be any one of a number of suitable liquid impermeable materials known in the art for use as outer covers or baffles in absorbent articles. Preferably, the baffle 30 will permit the passage of air and moisture vapor out of the pad 18 while blocking the passage of body fluids. A suitable material is a micro-embossed polymeric film, such as polyethylene or polypropylene, having a thickness of about 0.025 to 0.13 millimeters. Bicomponent films can also be used, as well as woven and nonwoven fabrics which have been treated to render them liquid impermeable. A specific example of a baffle material is a polyethylene film such as that used in KOTEX® pantiliners and obtainable from Pliant Corporation, Schaumburg, Ill., USA. The cover can be maintained in secured relation with the absorbent structure 32 by bonding all or a portion of the adjacent surfaces to one another. A variety of bonding methods known to one of skill in the art can be utilized to achieve any such secured relation. Examples of such methods include, but are not limited to, ultrasonic bonding, thermal bonding, or the application of adhesive materials in a variety of patterns between the two adjoining surfaces.

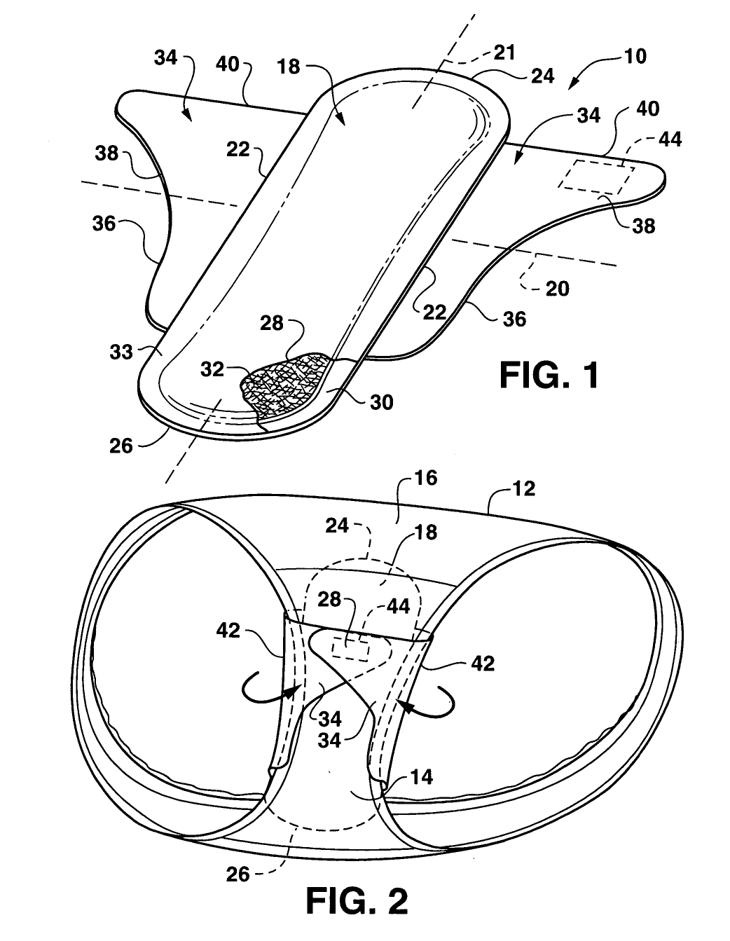

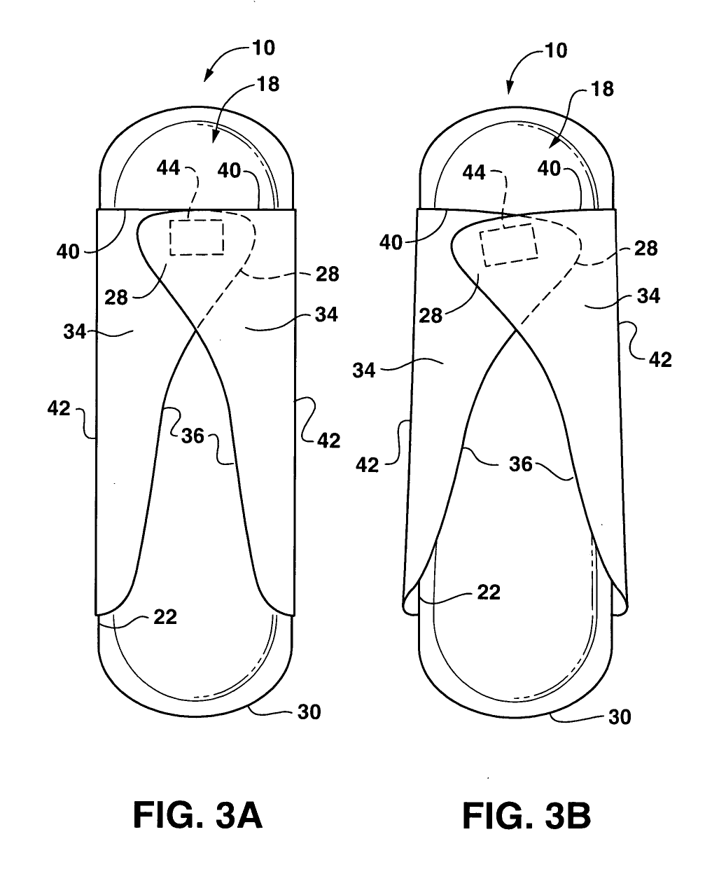

Referring to the figures in general, the article 10 includes wing portions 34 that extend laterally outward from the lateral sides 22 of the pad 18. The wing portions 34 can take on various shapes and dimensions to achieve their function of extending around the crotch region of a wearer's undergarment. Referring to FIG. 2, for example, the article 10 of FIG. 1 is illustrated attached to the crotch region 14 of a typical undergarment 12 to aid in securing the article 10 to the undergarment, as is generally understood by those skilled in the art. The area of the undergarment 12 designated as 16 is understood to be the front region of the undergarment. The wing portions 34 may be extensions of a single unitary structure attached to the baffle 30 with opposite laterally extending flaps that define the wing portions 34. In an alternate embodiment, each wing portion 34 may be a separate flap or panel attached to the baffle 30. In still another embodiment, the wing portions 34 may be defined by extensions of the baffle 30.

The wing portions 34 may be formed of the same material as the baffle 30. However, liquid impermeability and breathability are generally not concerns with the wing portions 34 and, thus, a different less expensive material may also be used. It should be appreciated that the type of wing material is not a limiting feature of the invention, and any suitable material may be used in this regard.

Referring to FIG. 1, the wing portions 34 have laterally outboard regions 38 that overlap when the wings 34 are folded around the crotch region 14 of the wearer's undergarment 12, as illustrated in FIG. 2. For this purpose, an attachment mechanism is provided to secure the overlap regions 38 to each other or to the garment material. The attachment mechanism may be any conventional device, such as an adhesive patch or strip that may be covered by a peel strip prior to use. The adhesive patch may have any shape, surface area, etc. The dashed region 44 in the figures is meant to identify the location or placement of any suitable attachment mechanism, and not to limit the size, shape, or type of mechanism. For placement of the article 10, the wearer simply peels the protective strip from the adhesive patch, places the article 10 in the crotch region 14 of the undergarment 12, folds the wing portions 34 around the crotch region 14, and presses the overlap regions 38 together such that the regions 38 attach to each other. In an alternate embodiment, or in addition to an adhesive strip, the attachment mechanism on the wing portions may include a mechanical fastener, such as a hook-and-loop fastener. For example, the patch 44 may be a patch of hook material that fastens to a loop-like material used to form the wing portions 34, such as a non-woven material. Alternately, a hook material may be placed on one or both of the wing portions 34 such that the wings attach directly to the undergarment material. Examples of hook-and-loop fastening systems and components are described in U.S. Pat. No. 5,019,073 issued May 28, 1991 to T. Roessler et al., the entire disclosure of which is hereby incorporated by reference in a manner that is consistent herewith. Other examples of hook-and-loop fastening systems are described in U.S. patent application Ser. No. 366,080 entitled HIGH-PEEL TAB FASTENER, filed Dec. 28, 1994 by G. Zehner et al. which corresponds to U.S. Pat. No. 5,605,735; and U.S. patent application Ser. No. 421,640 entitled MULTI-ATTACHMENT FASTENING SYSTEM, filed Apr. 13, 1995 by P. VanGompel et al.; the entire disclosures of which are hereby incorporated by reference in a manner that is consistent herewith. Examples of fastening tabs constructed with a carrier layer are described in U.S. patent application Ser. No. 08/603,477 of A. Long et al., entitled MECHANICAL FASTENING SYSTEM WITH GRIP TAB and filed Mar. 6, 1996 which corresponds to U.S. Pat. No. 5,624,429 which issued Apr. 29, 1997, the entire disclosure of which is hereby incorporated by reference in a manner which is consistent herewith. It should be appreciated that various attachment device configurations are within the scope and spirit of the invention.

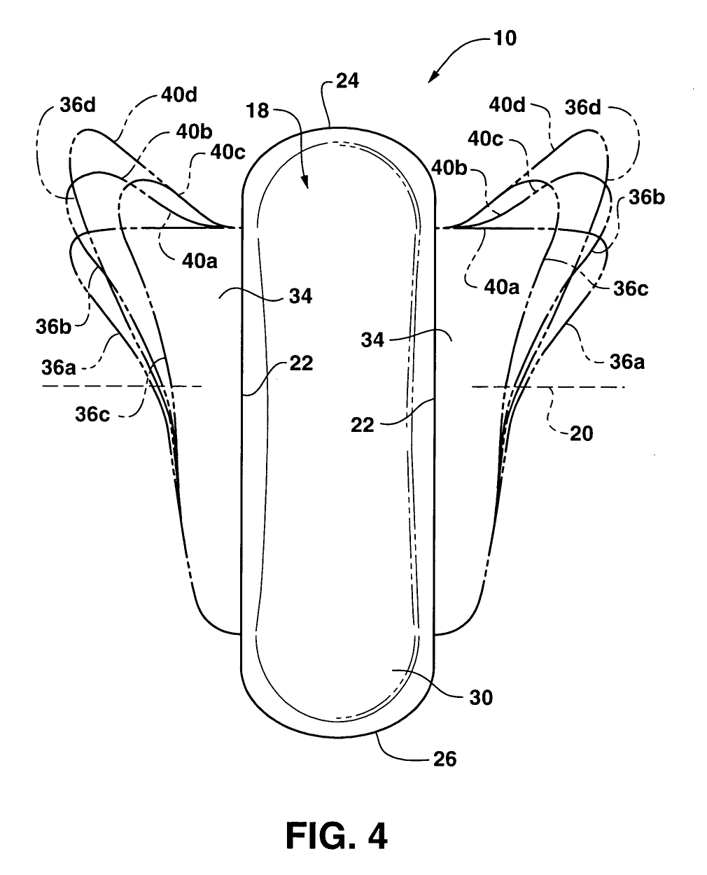

The wing portions 34 extend longitudinally along both sides of a transverse centerline 20 of the pad 18, as seen particularly in FIGS. 1 and 4. In a particular embodiment, the wing portions 34 extend along a substantial portion of the lateral sides 22 of the pad 18, for example along about two-thirds of the length of the sides 22, and may extend an equal distance on each side of the centerline 20. The wing portions 34 have an asymmetric shape with the overlap regions 38 disposed forward of the transverse centerline 20 towards the forward longitudinal end 24 of the pad 18. With this configuration, referring to FIG. 2, the article 10 may be placed in the desired middle or lowermost part of the undergarment's crotch region 14 with the overlap regions 38 secured closer to a front waist region 16 of the undergarment 12. As described above, the asymmetric configuration of the wing portions 34 with the overlap regions 38 disposed entirely on the forward end side of the transverse centerline 20 offers substantial benefits. For example, the wing portions 34 may be attached to the undergarment 12 or to each other at a wider part of the crotch region 14, thus providing a more secure attachment of the article. Also, the location of the wings 34 provides a visual indication to the wearer of the correct orientation of the article for use.

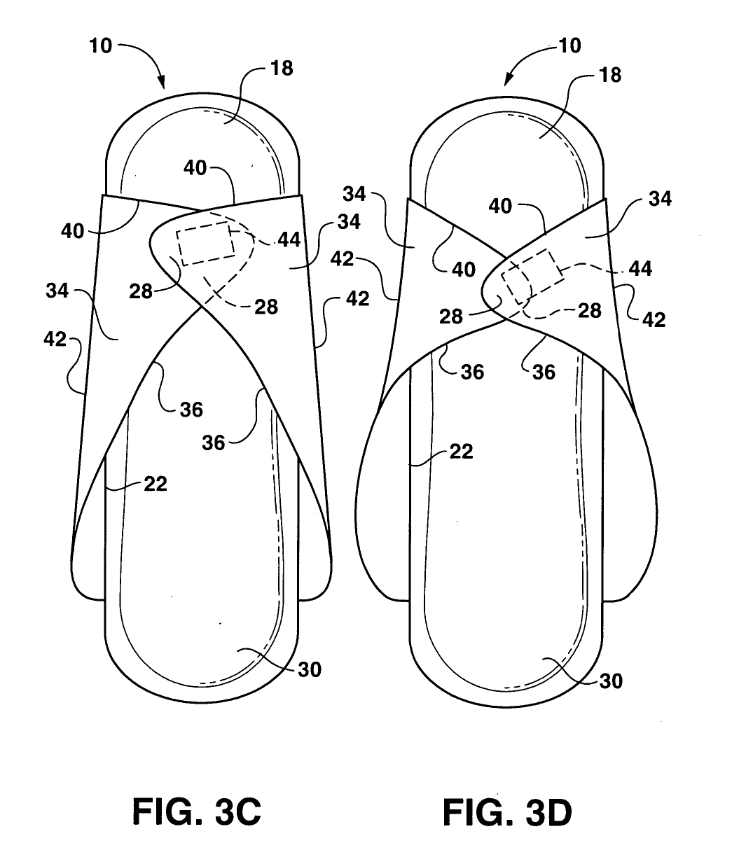

The asymmetric shape of the wing portions 34 may take on various shapes. In the illustrated embodiment, the wing portions have a forward most laterally extending edge 40 and a generally tapering outboard edge 36. The edge 36 tapers from the overlap region 38 towards the lateral side 22 of the pad 18. The edge 36 may taper at a constant angle, a variable angle, step-wise, and so forth. This unique configuration also allows for variable fastening or fold angles of the wing portions 34 with respect to the lateral sides 22 of the pad 18, as seen in FIGS. 3A through 3D. In FIG. 3A, it can be seen that the wing portions 34 are folded such that the fold lines 42 are generally parallel to the pad lateral sides 22 and the forward edge 40 is generally perpendicular to the sides 22. This fold pattern may be desired if the undergarment has a fairly wide and constant width crotch region 14, as in the undergarment 12 shown in FIG. 2. FIGS. 3B through 3D shown embodiments wherein the fold lines 42 are increasingly angled away from the lateral sides 22 such that the forward edges 40 are drawn towards the center of the pad 18 and the rearward portions of the wings flare outwardly. These different fold patterns may be desired for various styles and configurations of undergarments. FIG. 3D depicts a generally maximum angle of the fold lines 42. Even in this configuration, the overlap regions 38 are at least partially overlapping and disposed forward of a transverse centerline 20 (FIG. 1) of the pad 18.

It should also be appreciated that the overlap regions 38 may be configured such that the wing portions 34 are folded along fold lines 42 that angle towards the lateral sides 22 from the front of the pad to the back of the pad. In this embodiment, the frontward portions of the wings would flare outwardly, generally opposite to the embodiment of FIGS. 3A-3D. To accommodate this fold pattern, the overlap regions 28 would need an lateral extension and shape so as to be drawn towards the front of the pad in the various fold patterns, and not towards the center of the pad as with the embodiments of FIGS. 3A-3D.

FIG. 4 illustrates other various embodiments of the wing portions 34. For example, with one embodiment, the wing portions 34 have forward most edges 40a that are generally perpendicular to the pad lateral sides 22, and outboard tapered edges 36a such that the wing portion assumes an overall right triangle-type configuration. In an alternate embodiment, the wing portions 34 have outboard edges 36b and forward most edges 40b extending laterally from the sides 22 at an angle towards the forward end 24. In still another embodiment, the wing portions 34 extend laterally to a lesser extent and have angled forward most edges 40c and outboard edges 36c. In the final illustrated embodiment, the wing portions 34 have angled forward most edges 40d that extend longitudinally past the end 24 of the pad 18.

It should be appreciated by those skilled in the art that various modifications and variations can be made to the embodiments of the absorbent article described herein without departing from the scope and spirit of the invention as set forth in the appended claims and equivalents thereof.

1. An absorbent article for placement in the crotch region of an undergarment, comprising:
a pad component, said pad component further comprising
a generally liquid permeable top cover;
a generally liquid impermeable baffle;
an absorbent structure disposed between said top cover and said baffle;
protective wing portions extending laterally outward from lateral sides of said pad component, said wing portions having dimensions so as to wrap at least partially around the crotch portion of the undergarment;
said wing portions extending longitudinally along both sides of a transverse centerline of said pad and comprising an asymmetric shape with an overlap region disposed forward of a transverse centerline of said pad.

2. The absorbent article as in claim 1, wherein said wing portions comprise an outboard edge that tapers generally from said overlap region towards said lateral side of said pad.

3. The absorbent article as in claim 1, wherein said overlap regions are disposed at a forward most position on said wing portions.

4. The absorbent article as in claim 3, wherein said wing portions comprise a forward most edge that extends generally perpendicular from said lateral side of said pad and defines said overlap regions.

5. The absorbent article as in claim 3, wherein said wing portions comprise a forward most edge that extends at an angle from said lateral side of said pad towards a forward most longitudinal end of said pad.

6. The absorbent article as in claim 1, wherein said wing portions are foldable along laterally inboard fold lines that are variably positionable relative to said pad lateral sides while maintaining an overlapping relationship between said overlap regions.

7. The absorbent article as in claim 6, wherein said fold lines are angled away from said pad lateral sides from a forward most point of said fold lines to a rearward most point of said fold lines.

8. The absorbent article as in claim 6, wherein said fold lines are angled towards said pad lateral sides from a forward most point of said fold lines to a rearward most point of said fold lines.

9. The absorbent article as in claim 1, further comprising an attaching mechanism between said overlap regions of said wing portions.

10. The absorbent article as in claim 1, wherein said article is one of a sanitary napkin, panty liner, and adult incontinent article.

11. An absorbent article for placement in the crotch region of an undergarment, comprising:
a pad component, said pad component further comprising
a generally liquid permeable top cover;
a generally liquid impermeable baffle;
an absorbent structure disposed between said top cover and said baffle;
protective wing portions extending laterally outward from lateral sides of said pad component, said wing portions having dimensions so as to wrap at least partially around the crotch portion of the undergarment;
said wing portions extending longitudinally on both sides of a transverse centerline of said pad;
said wing portions further comprising laterally outboard regions that overlap in a folded configuration of said wing portion around the crotch region of an undergarment, said overlap regions disposed at a forward position entirely on one side of said transverse centerline;
said wing portions foldable along laterally inboard fold lines that angle away from said pad lateral sides from a forward most point of said fold lines to a rearward most point of said fold lines; and
wherein in a maximum angled position of said fold lines such that said overlap regions are maintained at least partially overlapping, said overlap regions remain entirely on said one side of said transverse centerline.

12. The absorbent article as in claim 11, wherein said wing portions comprise an asymmetric shape.

13. The absorbent article as in claim 11, wherein said wing portions extend at least about two-thirds along said lateral sides of said pad.

14. The absorbent article as in claim 11, wherein said wing portions comprise a forward most edge that extends generally perpendicular from said lateral side of said pad.

15. The absorbent article as in claim 11, wherein said wing portions comprise a forward most edge that extends at an angle from said lateral side of said pad towards a forward most longitudinal end of said pad.

16. The absorbent article as in claim 11, further comprising an attaching mechanism between said overlap regions of said wing portions.

17. The absorbent article as in claim 11, wherein said article is one of a sanitary napkin, panty liner, and adult incontinent article.
